(19) United States
(12) Patent Application Publication
(10) Pub. No.: US 2026/0090908 A1
(43) Pub. Date: Apr. 2, 2026

(54) PERSONAL TEMPERATURE REGULATING DEVICE AND METHODS OF USE THEREOF

(71) Applicant: Frostbite Industries Inc., Spring, TX (US)

(72) Inventors: Michael Leland FROST, Spring, TX (US); Adam Jacob FROST, Spring, TX (US)

(21) Appl. No.: 19/340,477

(22) Filed: Sep. 25, 2025

Related U.S. Application Data
(60) Provisional application No. 63/701,269, filed on Sep. 30, 2024.

(51) Int. Cl. A61F 7/00; A61F 7/02

(52) U.S. Cl. CPC A61F 7/007; A61F 2007/0093; A61F 2007/0096; A61F 2007/0234

(57) ABSTRACT

A personal temperature regulating device, comprising: a wearable garment and a temperature regulating subassembly coupled to the wearable garment. The temperature regulating subassembly comprising a temperature regulating membrane and a control module. The temperature regulating membrane comprising at least one temperature regulating subcomponent, wherein the at least one temperature regulating subcomponent is configured to alter a body temperature of a user.

### CROSS-REFERENCE TO RELATED APPLICATION

This application claims the benefit of U.S. Provisional Application No. 63/701,269, filed September 30, 2024, which is incorporated by reference herein in its entirety.

### BACKGROUND

### Field

The present disclosure generally relates to a personal temperature regulating device and methods of use thereof.

### Description of the Related Art

Personal temperature regulating devices are used to control and regulate the body temperature of a person using or wearing said device. The person using or wearing said device oftentimes uses these devices to heat up their body temperature or cool-down their body temperature. Occasionally the desire to cool or heat body temperature is for safety and/or comfort reasons. For instance, persons working outside in the heat, such as at a construction site during the summer, who may need to avoid heat stroke, discomfort and other heat-related conditions need a way to cool their body temperature down. Alternatively, persons working outside in the cold may need to avoid hypothermia and other cold-related conditions. Sometimes, the desire to cool or heat body temperature is for comfort reasons. Currently temperature regulating devices may either heat or cool wearers and/or users. For example, current temperature regulating devices may cool by, for example, chemical reactions in ice packs or similar components. Similarly, current temperature regulating devices may heat by, for example, chemical reactions such as those utilized in heat packs or electrical power. However, the current regulating devices lack methods of both heating and cooling. Furthermore, current devices and methods for cooling are cumbersome, inefficient, and less effective than desired and current devices and methods for heating are cumbersome, inefficient, and less effective than desired.

Thus, there is a need in the art for improved personal temperature regulating devices and methods of use thereof.

### SUMMARY

According to one or more embodiments, a personal temperature regulating device, comprising: a wearable garment and a temperature regulating subassembly coupled to the wearable garment. The temperature regulating subassembly comprising a temperature regulating membrane and a control module. The temperature regulating membrane comprising at least one temperature regulating subcomponent, wherein the at least one temperature regulating subcomponent is configured to alter a body temperature of a user.

According to one or more embodiments, a temperature regulating membrane, comprising a plurality of temperature regulating subcomponents, wherein the plurality of temperature regulating subcomponents are flexibly mechanically interconnected. The plurality of temperature regulating subcomponents each comprising: a heat sink, a plurality of bottom bus bars disposed in the heat sink, a plurality of semiconductive members disposed on the plurality of bottom bus bars, a semiconductive member support disposed about the plurality of semiconductive members, a plurality of top bus bars disposed on the plurality of semiconductive members, a frame, and a top plate. Each of the plurality of the bottom bus bars sits below and couples two of the plurality of semiconductive members. Each of the plurality of top bus bars sits atop and couples two of the plurality of semiconductive members. The plurality of top bus bars are disposed in the frame, and wherein the frame secures the plurality of top bus bars atop the plurality of semiconductive members. The top plate covering the plurality of top bus bars.

According to one or more embodiments, a temperature regulating subcomponent, comprising: a heat sink, a plurality of bottom bus bars disposed in the heat sink, a plurality of semiconductive members disposed on the plurality of bottom bus bars, a semiconductive member support disposed about the plurality of semiconductive members, a plurality of top bus bars disposed on the plurality of semiconductive members, a frame, and a top plate. Each of the plurality of the bottom bus bars sits below and couples two of the plurality of semiconductive members. Each of the plurality of top bus bars sits atop and couples two of the plurality of semiconductive members. The plurality of top bus bars are disposed in the frame, and wherein the frame secures the plurality of top bus bars atop the plurality of semiconductive members. The top plate covering the plurality of top bus bars.

According to one or more embodiments, a method regulating the temperature of a user with a personal temperature regulating device comprising: detecting a body temperature of a user wearing the personal temperature regulating device with a sensor of the personal temperature regulating device; determining the body temperature is different than a setting, wherein the setting is set by a control module of the personal temperature regulating device; and supplying an amount of power into one or more temperature regulating subcomponents of a temperature regulating membrane of the personal temperature regulating device, wherein the amount of power is based on the difference of the body temperature and the setting, and wherein the amount of power alters a surface temperature of a first side of the one or more temperature regulating subcomponents to alter the body temperature of the user.

The following description and the appended figures set forth certain features for purposes of illustration.

### BRIEF DESCRIPTION OF DRAWINGS

So that the manner in which the above-recited features of the disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 illustrates a user wearing a personal temperature regulating device, according to one or more embodiments.

FIG. 2 illustrates the personal temperature regulating device of FIG. 1, according to one or more embodiments.

FIG. 3 is a detailed view of the personal temperature regulating device of FIGS. 1-2, according to one or more embodiments.

FIG. 4 is a schematic view of a temperature regulating subassembly of the personal temperature regulating device of FIGS. 2-3, according to one or more embodiments.

FIG. 5A is a perspective view of a temperature regulating subcomponent for use in the temperature regulating membrane of FIG. 4, according to one or more embodiments.

FIG. 5B is a cross-sectional view of the temperature regulating subcomponent of FIG. 5A, according to one or more embodiments.

FIG. 5C is a top view of an exemplary electrical path through the temperature regulating subcomponent of FIGS. 5A-5B, according to one embodiment.

FIG. 6A is a perspective view of another temperature regulating subcomponent for use in the temperature regulating membrane of FIG. 4, according to one or more embodiments.

FIG. 6B is a cross-sectional view of the temperature regulating subcomponent of FIG. 6A, according to one or more embodiments.

FIG. 6C is a side view of a frame of the temperature regulating subcomponent of FIG. 6A, according to one or more embodiments.

FIG. 6D is a top view of a first portion of the frame of FIG. 6C, according to one or more embodiments.

FIG. 6E is a top view of a second portion of the frame of FIG. 6C, according to one or more embodiments.

FIG. 6F is a top view of a third portion of the frame of FIG. 6C, according to one or more embodiments.

FIG. 6G is a perspective view of the frame of FIG. 6C including the bottom bus layer, the top bus layer, and the semiconductive layer, according to one or more embodiments.

FIG. 6H is a perspective view of the top bus layer, the semiconductive layer, and the bottom bus layer, according to one or more embodiments.

FIG. 7 illustrates a method for regulating the temperature of a user with a personal temperature regulating device, according to one or more embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Aspects of the present disclosure provide systems, apparatus, and methods to a personal temperature regulating device for regulating the body temperature of a user and/or wearer. In one or more embodiments, the personal temperature regulating device may be a wearable personal temperature regulating device. The personal temperature regulating device includes a wearable garment and a temperature regulating subassembly. The temperature regulating subassembly may include a temperature regulating membrane, a control module, a power supply, and other components such as sensors and airflow devices. The temperature regulating membrane may include temperature regulating subcomponents that are mechanically interconnected, controlled by the control module, and/or powered by the power supply to regulate the body temperature of the user (e.g., heat or cool the user).

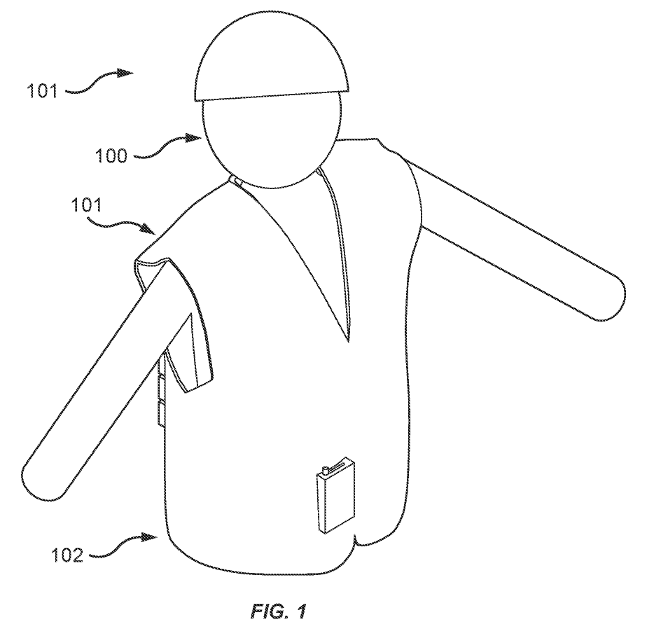

FIG. 1 illustrates a person (e.g., a user) 100 wearing coverings 101. In one or more embodiments, said coverings are personal protective equipment (PPE). The coverings 101 include a personal temperature regulating device 102. The personal temperature regulating device 102 may be worn and/or used to regulate the body temperature of the user 100 (e.g., heat or cool the user). For example, the user 100 may work or conduct activities outside. Such work or activities may expose the user 100 to extreme (or merely uncomfortable or undesirable) temperatures. Such temperatures may be hot (e.g., above 70 °F) or cold (e.g., below 70 °F) or somewhere therebetween. In such instances, the user 100 may desire to regulate their body temperature for comfort or safety. For example, a user 100 who is a construction worker may need to regulate their body temperature for outdoor work during peak heat during the summer or peak cold during the winter. As another example, a user 100 may want to use the personal temperature regulating device 102 to merely be comfortable inside when they are too cold or are too hot. As another example, a user 100 may want to utilize a personal temperature regulating device while operating a motor vehicle where cooling or heating is required (e.g., a motorcycle or tractor or some other vehicle without sufficient heating or cooling capabilities).

While the personal temperature regulating device 102 is illustrated and described above as PPE, the personal temperature regulating device 102 may be used and/or worn by a user 100 for leisure outside of the working environment. For instance, the personal temperature regulating device 102 may be worn by the user 100 as one or more pieces of clothing (including, but not limited to, vests, overalls, coveralls, upper body-wear, short-sleeved shirts, long-sleeved shirts, lower body-wear, pants, long pants, short pants, outerwear, jackets, coats, dress-wear, athletic wear, headwear, footwear, hand-wear, waist/fanny pack and backpack). Further, the personal temperature regulating device 102 may be used by a user 100 as simply a covering such as a blanket, a temperature regulation pad, or a temperature regulation patch. In one or more embodiments, the personal temperature regulating device 102 need not be worn. Rather, the personal temperature regulating device 102 may be incorporated into other things that may contact the body of a user 100. Non-limiting examples include furniture, such as chairs, couches, seats, and beds, handheld items, and other products that require some degree of high efficiency or flexibility and comfort.

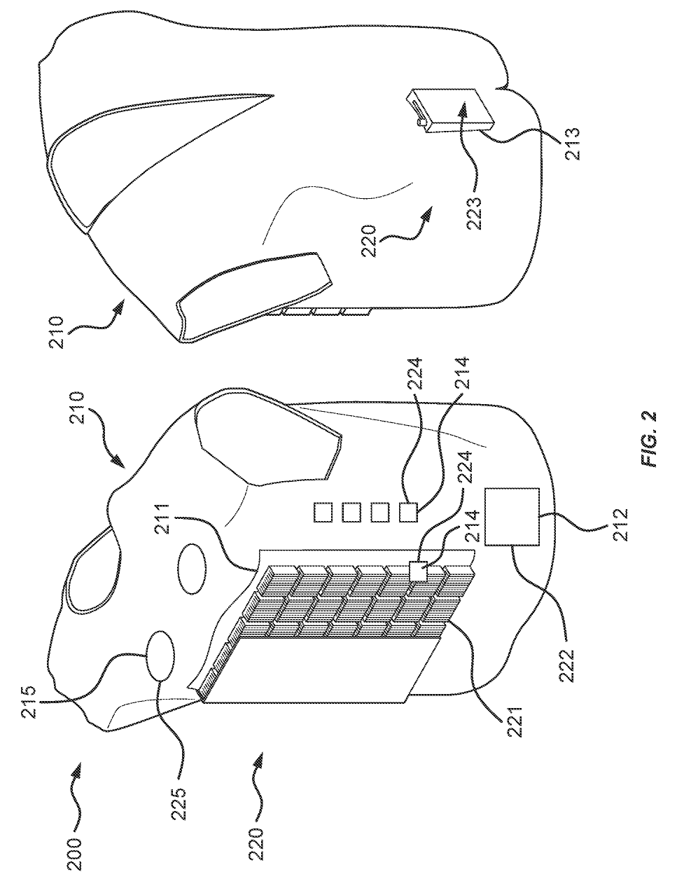

FIG. 2 illustrates an exemplary personal temperature regulating device 200. Exemplary personal temperature regulating device 200 may be used in place of personal temperature regulating device 102 of FIG. 1. That is, the personal temperature regulating device 200 shown in FIG. 2 is a non-limiting example of the personal temperature regulating device 102 of FIG. 1.

The personal temperature regulating device 200 includes a garment 210 and a temperature regulating subassembly 220.

The temperature regulating subassembly 220 is configured to regulate the body temperature of a user (such as user 100 of FIG. 1) by regulating the body temperature (e.g., heating or cooling) of the user through interactions with the garment 210 and the body of the user through the garment 210. The regulation of body temperature is also controllable by the user through the temperature regulating subassembly 220. The temperature regulating subassembly 220 includes a temperature regulating membrane 221, a power supply 222, a control module 223, sensors 224, and, optionally, one or more airflow devices 225 (e.g., fans).

As previously discussed with regards to FIG. 1, the garment 210 includes anything that may be worn or draped over the user and may include, but is not limited to, one or more pieces of clothing (including, but not limited to, vests, overalls, coveralls, upper body-wear, short-sleeved shirts, long-sleeved shirts, lower body-wear, pants, long pants, short pants, outerwear, jackets, coats, dress-wear, athletic wear, headwear, footwear, hand-wear, waist/fanny pack, and backpack), blankets, pads, and patches. As such, the garment 210 can include features such as sleeves, arm holes, hoods, head holes, pant legs, foot holes, zippers, buttons, elastic, pockets, a bill, finger holes, and/or anything typically used in clothing, blankets, patches, or other wearables. In one or more embodiments, the garment 210 may simply be a harness that may be strapped (potentially using but not limited to zippers, buttons, elastic, clips, or hook-and-loop fasteners) onto the user and may be used with or without other clothing or drapery. In one or more embodiments, the harness may be a wearable device not providing full coverage (e.g., only straps, such as shoulder straps, chest straps, waist straps) that the user 100 may wear underneath or above other clothing or drapery. In one or more embodiments, the harness may be worn over other fabric such as a breathable or sweat-wicking fabric, wherein the other fabric contacts the back of the user 100 and provides comfort and thermal conductivity to the user 100 between the harness including the temperature regulating subassembly 220 and the user 100.

Also as previously discussed, the temperature regulating subassembly 220 as shown may be used with something other than a garment 210, such as furniture, flooring, countertops, tables, handheld items, and more. In such embodiments, the temperature regulating subassembly 220 is configured to regulate the body temperature of a user (such as user 100 of FIG. 1) by regulating the body temperature (e.g., heating or cooling) of the user through interactions with whatever surface is contacting the body of the user.

The garment 210 may be made of materials typically used for clothing and/or coverings such as blankets or other wearables. Such materials include, but are not limited to, cloth, cotton, wool, flannel, polyester, and/or other synthetic fabrics.

The components of the temperature regulating subassembly 220 are coupled to the garment 210. In one or more embodiments, said components are individually coupled to the garment 210. In one or more embodiments, said components are coupled to the garment 210 as a group. The components of the temperature regulating subassembly 220 may be removably coupled to the garment 210 or may be integral to (or non-removable from) the garment 210. An example of removably coupling may include hook-and-loop fastening, fastening by buttons, zippers, or magnets. An example of integral-type coupling may be sewing in the temperature regulating subassembly 220 to the garment 210.

The garment 210 includes a temperature regulating membrane attachment point 211, a power supply attachment point 212, a control module attachment point 213, sensor attachment points 214, and airflow device attachment points 215.

The temperature regulating membrane 221 is coupled to the garment 210 by the temperature regulating membrane attachment point 211. The term membrane used herein is not intended to be limiting and may include a layer or assembly that may be flexible or rigid assembly of components that generally form a planar or semi-planar feature. The power supply 222 is coupled to the garment 210 by the power supply attachment point 212. The control module 223 is coupled to the garment 210 by the control module attachment point 213. The sensors 224 are coupled to the garment 210 by the sensor attachment points 214. The airflow devices 225 are coupled to the garment 210 by the airflow device attachment points 215. In one or more embodiments, the power supply 222 may be a battery (or other portable power supply) coupled to the garment. However, in one or more embodiments, power may be supplied via an external device (e.g., a power cord plugged into an outlet). In such embodiments, the power supply 222 may be a voltage converter. In one or more embodiments, the power supply 222 may be a combination of a battery and/or a voltage converter coupled to an external power source.

In one or more embodiments, the power supply attachment point 212 may be a pocket within the garment 210 and the power supply 222 may be freely disposed within the pocket or may be secured in the pocket by being sewn into the garment 210, or may be secured in the pocket by buttons, zippers, hook-and-loop fasteners, or by magnets. In one or more embodiments, the power supply attachment point 212 may be on an external or internal surface of the garment 210. The power supply attachment point 212 may be in any portion of the garment 210 so long as it allows for the other components of the temperature regulating subassembly 220 to fit into/onto the garment 210 and a user to wear the garment 210. In one or more embodiments, the power supply attachment point 212 is near or around the hip of the user. In one or more embodiments, the power supply attachment point 212 is on the back portion of the garment 210 (e.g., where the back of the user is).

In one or more embodiments, the control module attachment point 213 may be a pocket within the garment 210 and the control module 223 may be freely disposed within the pocket or may be secured in the pocket by being sewn into the garment 210, or may be secured in the pocket by buttons, zippers, hook-and-loop fasteners, or by magnets. In one or more embodiments, the control module attachment point 213 may be a hook-and-loop fastener, adhesive, or magnet, or surface feature on the garment 210 allowing the control module 223 to be attached to the surface of the garment 210. The control module attachment point 213 may be in any portion of the garment 210 so long as it allows for the other components of the temperature regulating subassembly 220 to fit into/onto the garment 210 and the user to wear the garment 210. In one or more embodiments, the control module attachment point 213 is near or around the waist of the user or around or near the upper-torso of the user. In one or more embodiments, the control module attachment point 213 is on the front or side portion of the garment 210 (e.g., where the front or sides of the user are).

In one or more embodiments, the control module attachment point 213 and the power supply attachment point 212 may use clips, hooks, buttons, zippers, hook-and-loop fasteners or magnets to attach externally to a user’s body, waist, belt, or alternative article of clothing. In some embodiments, the attachment points 212, 213 may be a pocket disposed in an alternative article of clothing. In such embodiments wherein the control module 223 and power supply 222 are not directly attached to the garment 210, the control module 223 and power supply 222 are attached to the garment 210 by attachment to the temperature regulating membrane 221 (e.g.,by a wired connection)

In one or more embodiments, the sensor attachment points 214 may be pockets within the garment 210 and the sensors 224 and the sensors 224 may be freely disposed within the pockets or may be secured within the pockets by being sewn into the garment 210, or may be secured in the pocket by buttons, zippers, hook-and-loop fasteners, or by magnets. In one or more embodiments, the sensor attachment points 214 may be on the inside of the garment 210 so that the sensors 224 better read the user’s body temperature. In one or more embodiments, the sensor attachment points 214 may attach one or more of the sensors 224 to the temperature regulating membrane 221 to monitor the temperature of the temperature regulating membrane 221 on either or both sides of the membrane. Similarly, the sensors 224 may be part of, or disposed in, the temperature regulating membrane 221. These sensors 224 may allow for the temperature of the temperature regulating membrane 221 to take corrective action as necessary if the temperature regulating membrane 221 is determined to be too hot or too cold. In one or more embodiments, the sensor attachment points 214 may be a hook-and-loop fastener, adhesive, or magnet, or surface feature on the garment 210 allowing for the sensors 224 to be attached to the surface of the garment 210. The sensor attachment points 214 may be in any portion of the garment 210 so long as it allows for the sensing (e.g., detecting) of body temperature of the user and so long as other components of the temperature regulating subassembly 220 to fit into/onto the garment 210. In one or more embodiments, the sensors 224 are spaced out relatively evenly around the garment 210 (e.g., front and back and top and bottom and sides) to obtain an average body temperature reading. In one or more embodiments, the sensors 224 are local to a specific area of the garment 210 correlating to a particular point on the users body deemed important for monitoring of body temperature (e.g., near an artery, a particularly hot point on a body, near a particularly cold point on the body, near known points on a body that give off the most heat, and/or near known points on the body that absorb the most heat).

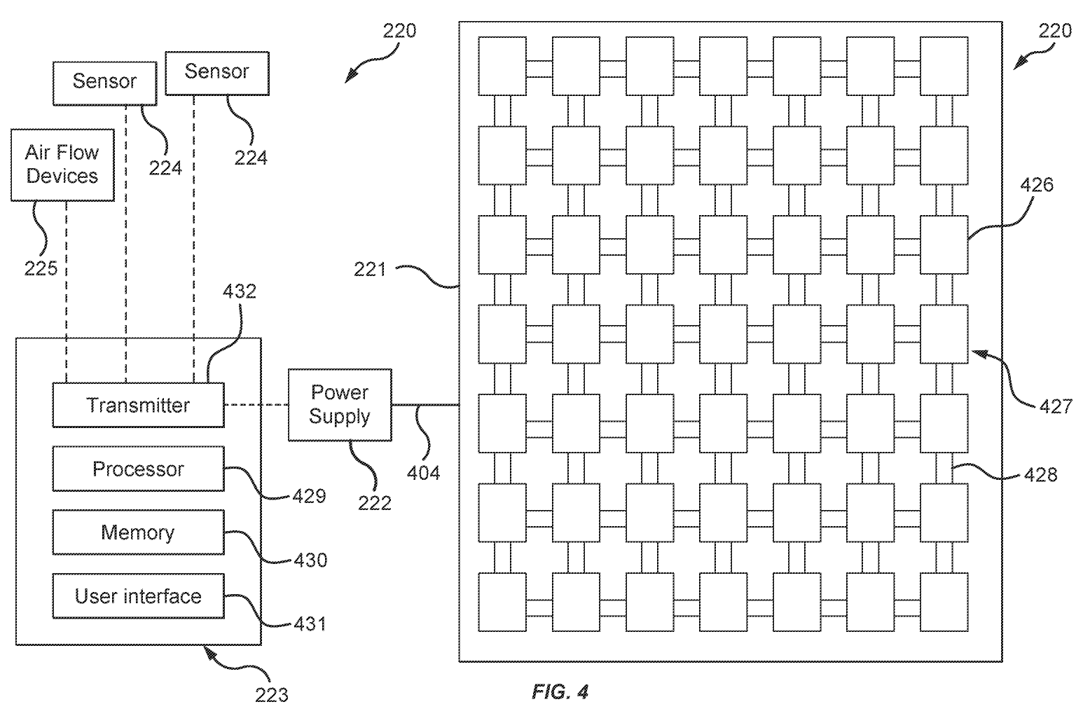

In embodiments including the airflow devices 225, the airflow device attachment points 215 may be pockets within the garment 210 and the airflow devices 225 may be freely disposed within the pockets or may be secured within the pockets by being sewn into the garment 210, or may be secured in the pocket by buttons, zippers, hook-and-loop fasteners, or by magnets. In one or more embodiments, the airflow device attachment points 215 may be a hook-and-loop fastener, or adhesive, or magnet, or surface feature on the garment 210 allowing for the airflow devices 225 to be attached to the surface of the garment 210. The airflow device attachment points 215 may be in any portion of the garment 210 so long as it allows for the airflow device 225 to flow air across the temperature regulating membrane 221. In one or more embodiments, the airflow device attachment points 215 may be near the bottom, top, sides, front, or back of the garment 210. Accordingly, no matter the location of the airflow device attachment point 215, the airflow devices 225 are configured, pointed, and/or angled to direct airflow across the temperature regulating membrane 221. In one or more embodiments, the airflow devices 225 may be integrated into the temperature regulating subassembly 220 rather than being separately attached to the garment 210. In one or more embodiments, the airflow devices 225 may be integrated into the temperature regulating membrane 221 as further described below with respect to FIG. 4.

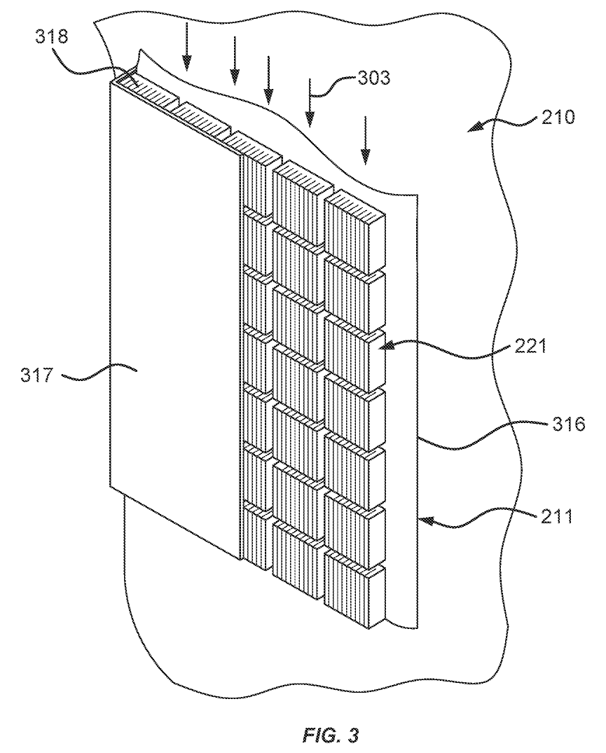

FIG. 3 is a detailed view of the personal temperature regulating device 200. In particular, FIG. 3 is a detailed view of the temperature regulating membrane attachment point 211. Although illustrated as being located on the back of the garment 210, the temperature regulating membrane attachment point 211 may be on the front, sides, top, bottom, inside, outside, or any other portion on the garment 210. In one or more embodiments, the temperature regulating membrane attachment point 211 may comprise attachment features at the edges 316 of the temperature regulating membrane attachment point 211. Such features may be zippers, buttons, hooks, hook-and-loop fasteners, thread (e.g., sewing the temperature regulating membrane 221 into place), clips, or elastic. In one or more embodiments, the attachment point 211 may include straps and/or other features configured to be strapped around or hung on a user, such as around the arms, shoulders, waist, chest, legs, arms, hands, or other body part of a user. In such embodiments, the straps and/or other features may be configured to attach the temperature regulating membrane 221 around the body of the user, outside of, or inside of, another article of clothing, rather than the garment 210 itself.

The temperature regulating membrane attachment point 211 may further comprise a cover flap 317. The cover flap 317 may be made of a flexible material, which may be the same material as the garment 210. In one or more embodiments, the cover flap 317 may be water proof in order to minimize liquid and solids exposure to the temperature regulating membrane 221.

The cover flap 317 may be attached to the garment 210 at one or more sides 318 of the temperature regulating membrane attachment point 211. The cover flap 317 at least partially covers and shrouds the temperature regulating membrane 221. One or more sides 318 of the cover flap 317 may not be attached to the garment 210 (e.g., at the top and bottom of the cover flap 317) such that airflow 303 is allowed to pass over the temperature regulating membrane 221. The one or more sides 318 of the cover flap 317 that are open may be on any side of the cover flap 317 so long as airflow is permitted over the temperature regulating membrane 221.

Thus, the cover flap 317 permits, shrouds, and directs airflow 303 over the temperature regulating membrane 221 to improve heat rejection by the temperature regulating membrane 221. The airflow 303 may be generated naturally or by airflow devices (such as airflow devices 225) which may be configured and pointed to direct airflow 303 through the open sides of the cover flap 317.

In one or more embodiments, the open sides of the cover flap 317 may include a material cover thus enclosing the temperature regulating membrane 221 while permitting airflow 303 and preventing particulate and/or foreign objects from coming into contact with the temperature regulating membrane 221, such as a mesh, netting, breathable, or perforated material. In one or more embodiments, the material enclosing the temperature regulating membrane 221 may be disposed outside of airflow devices 225 and thus enclose the airflow devices 225 with the temperature regulating membrane 221 (such as in embodiments wherein the airflow devices 225 are interconnected with the temperature regulating membrane 221).

FIG. 4 illustrates the temperature regulating subassembly 220 of the personal temperature regulating device 200. The temperature regulating subassembly 220 includes a temperature regulating membrane 221, a power supply 222, a control module 223, sensors 224, and, optionally, one or more airflow devices 225 (e.g., fans).

The temperature regulating membrane 221 includes one or more temperature regulating subcomponents 426. The one or more temperature regulating subcomponents 426 are flexibly mechanically interconnected and may be electrically interconnected. The temperature regulating membrane 221 includes an interface side 427 and a non-interfacing side (not shown). The interface side 427 is the side upon which the temperature regulating membrane 221 contacts a garment (such as garment 210 of FIGS. 2-3) or a user (such as user 100 of FIG. 1), or an intermediate component therebetween to transfer heat from or to the user.

There may be any number of temperature regulating subcomponents 426 in the temperature regulating membrane 221. For instance, in the embodiment illustrated in FIGS. 1-3, there are 35 temperature regulating subcomponents 426 arranged in a rectangular array of 5x7. Whereas in the embodiment illustrated in FIG. 4, there are 49 temperature regulating subcomponents 426 arranged in a rectangular array of 7x7. However, the temperature regulating subcomponents 426 may be arranged in any shape including but not limited to, a rectangle, circle, and a triangle. Furthermore, there may be greater than 35 temperature regulating subcomponents 426 and there may be less than 35 temperature regulating subcomponents 426. The array may also be of different widths and heights, for example, the array may be 2x2, 3x3, 4x4, 5x5, 6x6, 7x7 or 7x5, or any variation adding up to the total number of temperature regulating subcomponents 426. The number of temperature regulating subcomponents 426 is determined based on the packing density needed for efficiency of the temperature regulating membrane 221 and flexibility needed for the user and may be based on the heat transfer required change the body temperature of the user a desired amount (e.g., heat or cool the user). It may be beneficial to increase packing density (e.g., including more temperature regulating subcomponents 426 per unit of area) as you increase the number of temperature regulating subcomponents 426 per unit area, the more cooling can be accomplished in that unit of area. In one or more embodiments, the temperature regulating subcomponents 426 are spaced apart 1.0mm to about 500.0mm

The temperature regulating subcomponents 426 are flexibly mechanically interconnected to create the temperature regulating membrane 221. The mechanical, but flexible interconnection allows for use in garments, such as the examples illustrated above, that require the garment to conform to a user’s body for comfort or flexibility.

In one or more embodiments, such flexible mechanical interconnection may come from the temperature regulating subcomponents 426 being individually coupled to (e.g., sewn onto/into, adhesively coupled to, or hook-and-loop fastened to) a sheet of fabric, some other semi-flexible or flexible substrate or member, and/or the garment itself.

In one or more embodiments, such as the one illustrated, such flexible mechanical interconnection may come from elastic material 428 interconnecting each of the temperature regulating subcomponents 426 thus creating an interconnected array of the temperature regulating subcomponents 426 in a grid pattern. In one or more embodiments, each of the temperature regulating subcomponents 426 include a D-loop, G-loop, or similar type fitting on the one or more sides of the temperature regulating subcomponents 426, such as all four sides of the temperature regulating subcomponents 426, and the elastic material 428 interconnects the temperature regulating subcomponents 426 by the D-loop, G-loop, or similar fitting. In such embodiments, the fittings on the outer edge of the array of the temperature regulating subcomponents 426 may be used to attach (by elastic material 428 or otherwise) the temperature regulating membrane 221 to the garment (e.g., temperature regulating membrane attachment point 211). In one or more embodiments, the elastic material 428 may also be woven around the temperature regulating subcomponents 426 such as weaving over and under alternating temperature regulating subcomponents 426. In one or more embodiments, the elastic material 428 interconnects alternating temperature regulating subcomponents 426, such as by the D-fittings, such that every other temperature regulating subcomponent 426 is attached to one another by the elastic material 428. In one or more embodiments, independent pieces of elastic material 428 may wrap around one or more fittings to allow separate attachment and removal of independent temperature regulating subcomponents 426. However, it is to be understood that any temperature regulating subcomponent 426 may be interconnected to any other temperature regulating subcomponent 426.

The temperature regulating subcomponents 426 may be electrically connected to the power supply 222 so that the power supply 222 may supply power 404 (power in watts (W)). The temperature regulating subcomponents 426 may be connected to the power supply 222 in series or may be wired to the power supply 222 in parallel or some combination of series and parallel. Each of the temperature regulating subcomponents 426 may be individually wired to a bus which is wired to the power supply 222. Each of the temperature regulating subcomponents 426 may be wired directly to the power supply 222. Each of the temperature regulating subcomponents 426 may be wired to one another and the whole may be wired to the bus or the power supply 222. The power supply 222 may also provide power 404 to the remaining components of the temperature regulating subassembly 220.

According to one mode of operation, the power supply 222 supplies power 404 to the temperature regulating membrane 221. That is, the power supply 222 supplies power 404 to each of the temperature regulating subcomponents 426 causing the interface side 427 of the temperature regulating membrane 221 to heat or cool thus regulating the temperature of the body of the user. The amount of cooling and/or heating accomplished by the temperature regulating subcomponents 426 is determined by the amount of power 404 supplied to the temperature regulating membrane 221. As a non-limiting example, the temperature regulating membrane 221 may have a coefficient of performance (COP) of about 1.0 to about 10.0, such as between about 2.0 and about 5.0. For example, the temperature regulating membrane 221 may be supplied about 30W to about 35W of power 404 for about 80W of cooling. In another example, the temperature regulating membrane 221 may be supplied about 60W to about 80W of power 404 to about 160W to about 180W of cooling. In one or more embodiments, the power supplied may be about 0W to about 150W and the cooling power may be about 0W to about 300W or more.

Similarly, the power 404 supplied to the temperature regulating membrane 221 determines whether the interface side 427 is heating or cooling. In one or more embodiments, if the current of the power 404 supplied by the power supply 222 is positive (e.g., above zero), the interface side 427 of the temperature regulating membrane 221 is cooled. In the alternative, if the current of the power 404 supplied is negative (e.g., below zero), the interface side 427 of the temperature regulating membrane 221 is heated.

The power 404 supplied by the power supply 222 to the temperature regulating membrane 221 is controlled by the control module 223. The control module 223 allows the user to set a setting (e.g., a “cool” and “heat” setting, a “high,” “medium,” or “low” setting for both heating and cooling, a specific temperature setting, a range of numbers indicative of levels of cooling and/or heating such as 1-10, or a particular power level or amount). Based on the setting, the control module 223 controls the power 404 supplied by the power supply 222 to maintain or reach the desired setting. In one or more embodiments, the control module 223 may also control other components of the temperature regulating subassembly 220, such as the airflow devices 225. The control module 223 may include a processor 429, a memory 430, a user interface 431, and a transmitter 432.

The user interface 431 allows the user to control the temperature regulation. In other words, a user may use the user interface 431 to set the control module 223 to a desired setting. In one or more embodiments, the user interface 431 also allows a user or another to see the current body temperature of the user as determined by the one or more sensors 224, the current temperature membrane temperature, the current power level, or any other inputs or outputs of the temperature regulating subassembly 220.

In one or more embodiments, the user interface may include buttons, dials, a touch screen, a numerical display, or any combination thereof. In one or more embodiments, the control module 223 does not include a user interface 431 integral to the control module 223 and is instead controlled by an external device, such as a smart phone or external computing device. Said external device may operate as the user interface 431 allowing users, others, or an external computer system to set the desired setting remotely and see inputs and outputs remotely.

The processor 429 receives the setting from the user interface 431, retrieves instructions stored on the memory 430 based on the setting, and executes said instructions based on the settings. The instructions include, but are not limited to instructions to control the power supply 222 and the airflow devices 225. In one or more embodiments, the airflow devices 225 are additionally coupled to a voltage converter (e.g., a buck module) disposed between the power supply 222 and the airflow devices 225.

The transmitter 432 receives and sends instructions to components external to the control module 223. In one or more embodiments, the transmitter 432 may transmit and receive information by a wired connection to said external components. In one or more embodiments, the transmitter 432 may transmit and receive information by a wireless connection (e.g., Wi-Fi, Bluetooth or radio frequency) to said external components.

The transmitter 432 is configured to receive inputs and transmit outputs of the control module 223. For example, the transmitter 432 may receive inputs such as user body temperature readings or temperature regulating membrane interface side temperature readings from the sensors 224. Similarly, the transmitter 432 may transmit outputs, such as, instructions to the power supply 222 and airflow devices 225. In embodiments where an external device is used as a user interface 431, the transmitter 432 is configured to receive the setting information from the external device and may be configured to transmit outputs to the external device.

In one or more embodiments, the control module 223 includes its own power source. In one or more embodiments, the control module 223 is powered by the same power supply 222 used to supply power to the temperature regulating membrane 221.

The sensors 224 are configured to monitor temperature of the components of the temperature regulating subassembly 220 and the body temperature of the user. The sensors 224 are configured to monitor the body temperature of the user so that the reading may be used in a feedback loop for appropriate cooling and/or heating based on the desired setting and/or for safety and/or discomfort.

In one or more embodiments, the sensors 224 may also be configured to monitor the temperatures of the temperature regulating membrane 221. The sensors 224 are configured to monitor the temperature of the temperature regulating membrane 221 so that the reading may be used to prevent the temperature regulating membrane 221 from getting too hot or too cold.

The airflow devices 225 are configured to direct airflow over the temperature regulating membrane 221 to improve heat rejection by the temperature regulating membrane 221 in the cooling mode. Although illustrated as a separate component, as previously discussed, the airflow devices 225 may be integrated into the temperature regulating membrane 221. That is, the airflow devices 225 may be part of the flexible-mechanical interconnection of temperature regulating subcomponents 426. For example, the airflow devices 225 may include loops or openings or other coupling features that may be connectable to the elastic material 428 to integrate the airflow devices 225 into the temperature regulating membrane 221.

According to one mode of operation wherein the user wants to be cooled by the personal temperature regulating device 200, the user sets a corresponding setting with the user interface 431 of the control module 223, the processor 429 of the control module 223 retrieves corresponding instructions from the memory 430 of the control module 223, the processor 429 instructs, via the transmitter 432, the power supply 222 to supply a corresponding power 404 to the temperature regulating membrane 221, the power supply 222 supplies the corresponding power 404 to the temperature regulating membrane 221 and the temperature regulating subcomponents 426, and the temperature regulating subcomponents 426 cool the interface side 427 of the temperature regulating membrane 221 thus cooling the user. In one or more embodiments, the sensors 224 are utilized as a part of a feedback loop. Thus, while the power 404 is being supplied to the temperature regulating membrane 221 and the temperature regulating subcomponents 426, the sensors 224 monitor the body temperature of the user, the monitored body temperature is transmitted to the control module 223, and the control module 223 instructs the power supply 222 to adjust the power 404. The power 404 may be adjusted to increase cooling if the monitored body temperature is hotter than the desired setting or adjusts the power 404 to decrease cooling or increase heating if the monitored body temperature is colder than the desired setting.

According to one mode of operation wherein the user wants to be heated by the personal temperature regulating device 200, the user sets a setting with the user interface 431 of the control module 223, the processor 429 of the control module 223 retrieves corresponding instructions from the memory 430 of the control module 223, the processor 429 instructs, via the transmitter 432, the power supply 222 to supply a corresponding power 404 to the temperature regulating membrane 221, the power supply 222 supplies the corresponding power 404 to the temperature regulating membrane 221 and the temperature regulating subcomponents 426, and the temperature regulating subcomponents 426 heat the interface side 427 of the temperature regulating membrane 221 thus heating the user. In one or more embodiments, the sensors 224 are utilized as a part of a feedback loop. Thus, while the power 404 is being supplied to the temperature regulating membrane 221 and the temperature regulating subcomponents 426, the sensors 224 monitor the body temperature of the user, the monitored body temperature is transmitted to the control module 223, and the control module 223 instructs the power supply 222 to adjust the power 404. The power 404 may be adjusted to increase heating if the monitored body temperature is colder than the desired setting or adjusts the power 404 to decrease heating or increase cooling if the monitored body temperature is hotter than the desired setting.

The control module 223 may also control the heating and cooling of the temperature regulating membrane 221 for safe or comfortable usage of the temperature regulating subassembly 220 based on the body temperature of the user. For example, the sensors 224 monitor the body temperature of the user and if the control module 223 determines that the body temperature of the user is too hot (e.g., outside of a safety and/or comfort threshold) and indicative of overheating or discomfort, the control module 223 can instruct the power supply 222 to adjust power 404 accordingly. For example, the control module 223 can instruct the power supply 222 to supply a power 404 to cease heating, increase cooling, or shut off power supply 222. Similarly, if the control module 223 determines that the user’s body temperature is too cold (e.g., outside of a safety and/or comfort threshold) indicative of hypothermia or discomfort, the control module 223 can instruct the power supply 222 to adjust power 404 according. For example, the control module 223 can instruct the power supply 222 to supply an amount of power 404 to cease cooling, increase heating, or shut off power supply.

The control module 223 may also control the heating and cooling of the temperature regulating membrane 221 for safe and/or comfortable usage based on the temperature of the temperature regulating membrane 221. For example, the sensors 224 monitor the temperature of the temperature regulating membrane 221 and if the control module 223 determines that the temperature regulating membrane 221 is too hot for safe or comfortable use (e.g., outside of a safety or comfort threshold or operating in an unhelpful manner due to technical limitations), the control module 223 can instruct the power supply 222 to adjust accordingly. For example, the control module 223 can instruct the power supply 222 to supply an amount of power 404 to cease heating, increase cooling, or shut off power supply. Similarly, if the control module 223 determines that the temperature regulating membrane 221 is too cold for safe or comfortable use (e.g., outside of a safety and/or comfort threshold), the control module 223 can instruct the power supply 222 to adjust power 404 accordingly. For example, the control module 223 can instruct the power supply 222 to adjust an amount of power 404 to cease cooling, increase heating, or shut off power supply.

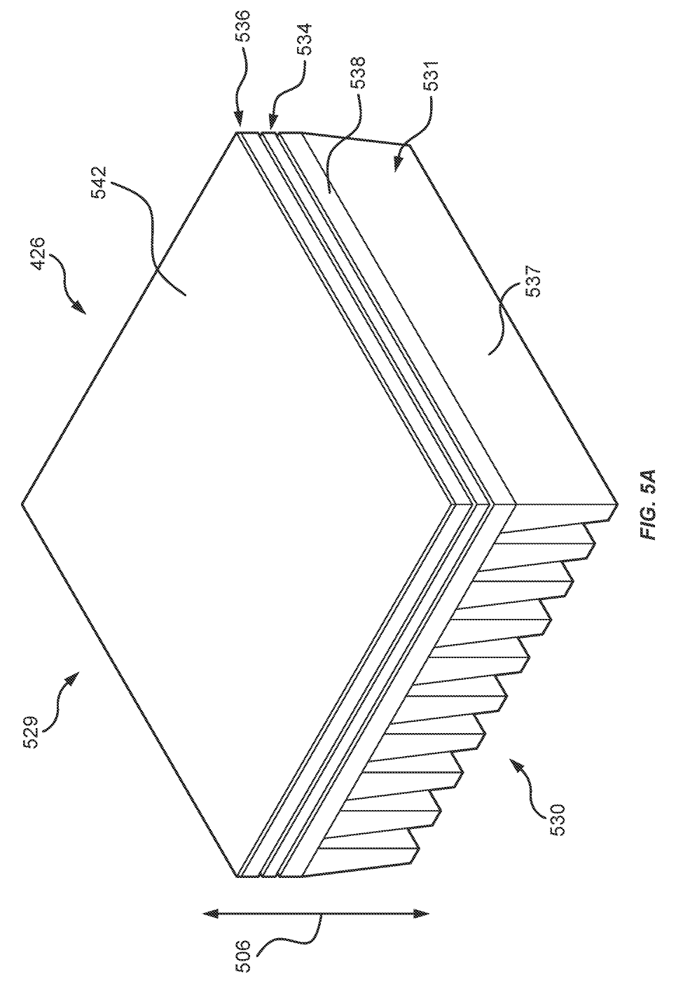

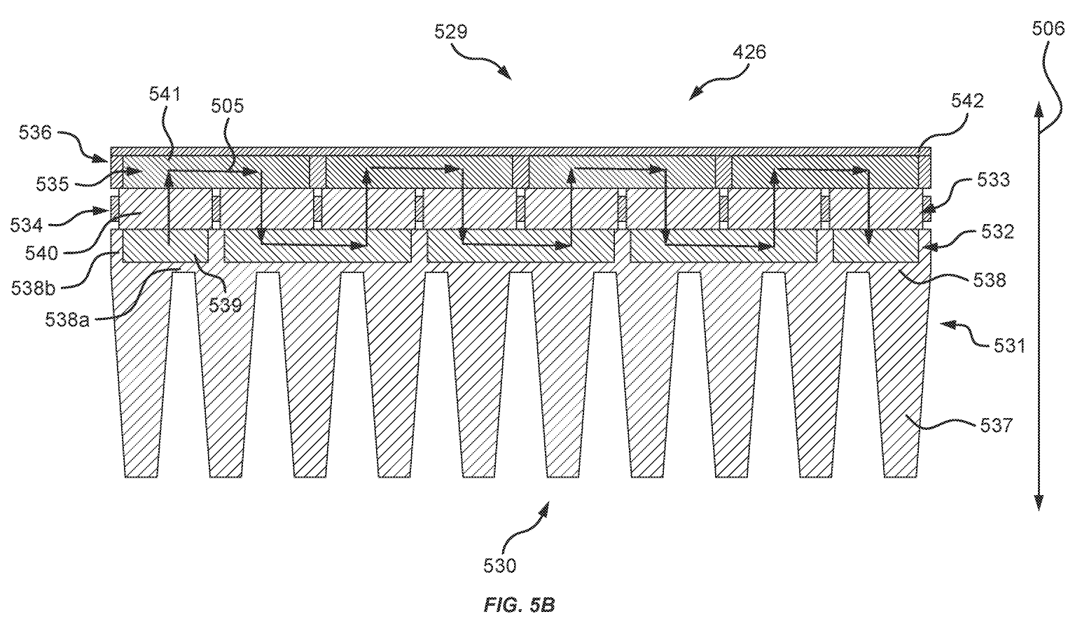

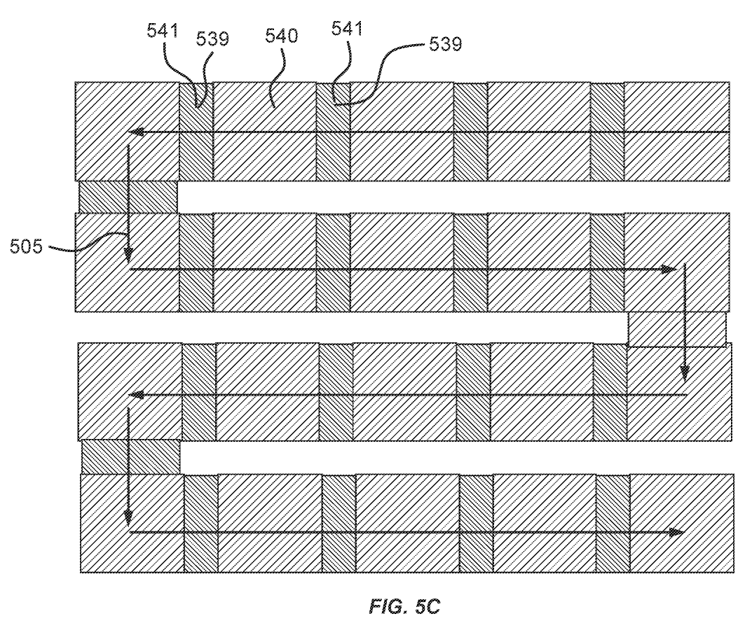

FIGS. 5A-5B illustrate a temperature regulating subcomponent 426. FIG. 5A illustrates a perspective view of the temperature regulating subcomponent 426. FIG. 5B illustrates a cross-sectional view of the temperature regulating subcomponent 426.

The temperature regulating subcomponent 426 may be a thermoelectric device. That is, the temperature regulating subcomponent 426 utilizes power supplied to the temperature regulating subcomponent 426 to transfer heat between opposite sides of the temperature regulating subcomponent 426 along heat path 506. In one or more embodiments, the temperature regulating subcomponent 426 may be about 10.0mm to about 100.0mm in width and about 10.0mm to about 100.0mm in length.

The temperature regulating subcomponent 426 includes a first side 529 and a second side 530. The first side 529 corresponds to an interface side (such as interface side 427 of FIGS. 4) of a temperature regulating membrane (such as temperature regulating membrane 221 of FIGS. 1-4). Thus, when the first side 529 is cooled, the interface side is cooled and when the first side 529 is heated, the interface side is heated.

The temperature regulating subcomponent 426 includes a heat sink 531, a bottom bus layer 532, semiconductive layer 533, a semiconductive member support 534, a top bus layer 535, a top bus support 536, and a top plate 542.

The heat sink 531 is disposed on the second side 530 of the temperature regulating subcomponent 426. The heat sink 531 includes fins 537 protruding from a heat sink base 538. The fins 537 extend the depth of the temperature regulating subcomponent 426 (e.g., into the page in FIG. 5B). The heat sink fins 537 allow for heat rejection and/or cold rejection into the environment around the second side 530 of the temperature regulating subcomponent 426. For instance, heat may be rejected by airflow (such as airflow 303 of FIG. 3) flowing over the fins 537. The heat sink 531 may include between 5 and 30 fins 537. For instance, in the illustrated embodiment, there are 10 fins 537. The fins 537 may have a generally trapezoidal, rectangular or hexagonal, cross section and may protrude about 1.0mm to about 20.0mm from the heat sink base 538. The fins 537 may have a minor width (the width nearest the tip of the fin 537) of about 0.1mm to about 5.0mm and a major width (the width nearest the base of the fin 537) of about 0.1mm to about 5.0mm. The space between the minor widths of the fins 537 may be about 0.1mm to about 5.0mm. The space between the major widths of the fins 537 may be about 0.1mm to about 5.0mm. In one or more embodiments, rather than having a heat sink base 538 with fins 537 having a generally rectangular or hexagonal cross section, heat sink 531 may have a sinusoidal cross section with similar dimensions to those described above. In one or more embodiments, the heat sink 531 may be made of an electrically resistive but thermally conductive material, or an electrically and thermally conductive material with a non-electrically-conductive coating, including, but not limited to, anodized aluminum, ceramic, or other coated metal. The heat sink may have full length or several short length fins, rectangular bars or fins of varying thickness (for durability).

In one or more embodiments, the heat sink base 538 may be made of two or more components, one of which components may have the fins 537 protruding from it, portion 538a, and the other component including a frame portion 538b configured to house the bottom bus layer 532. Together, portions 538a and 538b may make up the heat sink base 538.

The bottom bus layer 532 is disposed at least partially within the heat sink base 538. In embodiments wherein the heat sink base 538 includes separate frame portion 538b, the bottom bus layer 532 is at least partially disposed within the frame portion 538b of the heat sink base 538. The heat sink base 538 and the bottom bus layer 532 together act as a structural support for the temperature regulating subcomponent 426. The bottom bus layer 532 includes a plurality of bottom bus bars 539. The bottom bus bars 539 may be rectangular prisms or another shape. The bottom bus bars 539 are coupled to the heat sink base 538 by soldering. In one or more embodiments, the bottom bus bars 539 are coupled to the heat sink base 538 by an adhesive, such as an epoxy or glue, such as a thermal epoxy. In one or more embodiments, a layer of metal, such as copper or nickel, may be sputtered and/or otherwise coated on the heat sink base 538 and disposed between the bottom bus bars 539 and the heat sink base 538.

The semiconductive layer 533 includes semiconductive members 540. The semiconductive members 540 are arranged in an array atop the bottom bus layer 532 and coupled to the bottom bus layer 532 by soldering. In some embodiments, a coating of metal, such as nickel, may be sputtered and/or otherwise coated on the semiconductive members 540 between the semiconductive members 540 and the bottom bus layer 532 so that the solder may stick to the semiconductive members 540. The semiconductive members 540 may be made of a semiconductive material, such as bismuth telluride or materials doped with selenium or lead. The semiconductive members 540 may be shaped as a rectangular prism or may be another shape such as a cylinder or triangular prism. Accordingly, the semiconductive members 540 may have a height, width, and depth of between about 0.01mm and about 15mm, such as between about 1.0mm and about 10.0mm. The semiconductive members 540 are spaced apart from one another by about 0.01mm to about 2.0mm. There may be about 16 to about 500 semiconductive members 540 per temperature regulating subcomponent 426.

The semiconductive members 540 may be made of two types of semiconductive materials, P-type and N-type. As will be described herein, the P-type and N-type semiconductive members 540 are electrically coupled to one another by the bus bars 539, 541. The coupling of the P-type to the N-type semiconductive members 540 is what induces the temperature regulating effect of the temperature regulating subcomponent 426.

The bottom bus bars 539 described above electrically couple one of the semiconductive members 540 to another semiconductive member 540 (e.g., a P-type semiconductive member 540 to an N-type semiconductive member or vice versa). The bottom bus bars 539 each sit below two of the semiconductive members 540 (e.g., a P-type semiconductive member 540 and an N-type semiconductive member) allowing electricity to flow through one semiconductive member 540 and into another semiconductive member 540 (e.g.,a P-type semiconductive member 540 to an N-type semiconductive member or vice versa). In one or more embodiments, the bottom bus bars 539 move current from P-type semiconductive members 540 to N-type semiconductive members 540 Thus, the bottom bus bars 539 and semiconductive members 540 create an electrical path 505 through the temperature regulating subcomponent 426 between the semiconductive members 540. Accordingly, the number of bottom bus bars 539 depends on the number of semiconductive members 540. The bottom bus bars 539 may be made of a structural, electrically conductive, and heat conductive material including, but not limited to, copper. The bottom bus bars 539 may act as a structural support for the temperature regulating subcomponent 426. The bottom bus bars 539 also allow for heat transfer through the temperature regulating subcomponent 426 along heat path 506 due to their thermal conductivity.

The semiconductive member support 534 is disposed about the semiconductive layer 533 and about each semiconductive member 540 and is configured to arrange the semiconductive members 540 in the array such that there is a gap between each of the semiconductive members 540. Accordingly, the semiconductive member support 534 may be generally rectangular with cutouts sized and shaped to secure the semiconductive members 540 in which the semiconductive members 540 are disposed. The semiconductive member support 534 may be made of an electrically and thermally resistive material, including, but not limited to plastic. The electrical resistivity beneficially allows for the semiconductive members 540 to be electrically isolated from one another forcing electricity to flow along electrical path 505. The thermal resistivity also beneficially prevents heat from flowing backwards across the temperature regulating subcomponent 426.

The top bus layer 535 sits atop the semiconductive layer 533 and is disposed at least partially through the top bus support 536. The top bus layer 535 is coupled to the semiconductive layer 533 by soldering. In one or more embodiments, a thin coat of metal, such as nickel or copper, is sputtered and/or otherwise disposed on the semiconductive members 540 between the top bus layer 535 and the semiconductive layer 533 so that the solder sticks to the semiconductive members 540. The top bus layer 535 includes a plurality of top bus bars 541. The top bus bars 541 may be rectangular prisms or another shape. The top bus bars 541 electrically couple one of the semiconductive members 540 to another (e.g., a P-type semiconductive member 540 to an N-type semiconductive member or vice versa). The top bus bars 541 each sit atop two of the semiconductive members 540 (e.g., a P-type semiconductive member 540 to an N-type semiconductive member). In one or more embodiments, the top bus bars 541 move current from N-type semiconductive members 540 to P-type semiconductive members 540 allowing electricity to flow through one semiconductive member 540 and into another semiconductive member 540. Thus, the top bus bars 541, the semiconductive members 540, and the bottom bus bars 539 create the electrical path 505 through the temperature regulating subcomponent 426. Accordingly, the number of top bus bars 541 depends on the number of semiconductive members 540.

The orientation of the top bus bars 541 and bottom bus bars 539 determines the electrical path 505. One embodiment of the orientation of top bus bars 541 and bottom bus bars 539 is shown in FIG. 5C from a top view. FIG. 5B and FIG. 5C together illustrate the electrical path 505. According to said embodiment, and as shown in FIG. 5B, the electrical path 505 runs along a row of semiconductive members from one end of the temperature regulating subcomponent 426 to the other end. During this portion of the electrical path 505, the electricity is running from one bus bar 539, 541 to another bus bar 539, 541 through a semiconductive member 540. Once the electrical path 505 has reached the end that row of semiconductive members 540 and bus bars 539, 541, a bus bar 539, 541 is oriented perpendicularly to the other bus bars 539, 541 to pass the electrical path 505 to the next row of semiconductive members 540 and bus bars 539541. Thus, each of the semiconductive members 540 of the array are coupled to one another in series, creating electrical path 505.

The top bus bars 541 may be made of a structural, electrically conductive, and heat conductive material including, but not limited to copper. The top bus bars 541 may act as a structural support for the temperature regulating subcomponent 426. The top bus bars 541 also allow for heat transfer through the temperature regulating subcomponent 426 along heat path 506 due to their thermal conductivity.

The top bus support 536 encloses the top bus bars 541 and partially contains the top bus bars 541. The top bus bars 541 and the top bus support 536 together act as structural support for the temperature regulating subcomponent 426. The size and shape of the top bus support 536 corresponds to the size and shape of the temperature regulating subcomponent 426 (e.g., about 10.0mm to about 100.0mm by about 10.0mm to about 100.0mm). The size and shape of the spaces wherein the top bus bars 541 are disposed are based on the size and shape of the top bus bars 541. The top bus support 536 may be made of a thermally conductive but electrically resistive material including, but not limited to, anodized aluminum, ceramics, or another metal with an electrically resistive coating. This allows heat to be transferred through the top bus support 536 along heat path 506 without electricity conducting through the top bus support 536.

In one or more embodiments, the temperature regulating subcomponent includes one or more aluminum top plates 542 disposed on top of the top bus support 536 and coupled to the top bus support 536 by soldering. In one or more embodiments, the top plate 542 may be one or more layers of aluminum (e.g., two, three, four, five, or more). In one or more embodiments, a thin coat of metal, such as nickel or copper, is sputtered and/or otherwise disposed between the top bus layer 535 and the top plate 542. The top plate 542 covers the exposed portion of the top bus bars 541 so that the top bus bars 541 are not exposed to the environment and have an electrical barrier. The top plate 542 covers the top bus bars 541 so that two or more of the top bus bars 541 are not short circuited by something that contacts them. Such a short may occur from anything conductive, such as metal or sweat. In one or more embodiments, the top plate 542 may be an aluminum printed circuit board (PCB). In one or more embodiments, the aluminum PCB may be an anodized or coated aluminum plate with thin layers of conductive material sprayed or spattered on top of the aluminum plate.

The top plate 542 is made of a thermally conductive material but an electrically resistive material, such as anodized aluminum. The thermal conductivity allows for heat to transfer through the top plate 542. The electrical resistivity prevents shorting of the top bus bars 541. In one or more embodiments, the top plate 542 is also made from an electrically conductive material. In one or more embodiments, the electrically conductive material may be an aluminum PCB. In one or more of such embodiments, the bottom of the top plate 542 (e.g., the side facing the top bus bars 541) may be electrically insulating (such as by a coating or anodizing).

According to one mode of operation, power is supplied to a temperature regulating subcomponent 426, the current of the power runs through one of the semiconductive members 540 (e.g., from bottom to top or top to bottom) along electrical path 505, through a bus bar 539, 541, to another semiconductive member 540, through the second semiconductive member 540, through another bus bar 539, 541, and so on. Each time the electricity reaches each top bus bar 541, the first side 529 of the temperature regulating subcomponent 426 is either heated or cooled (depending on whether the current is positive or negative) and each time the electricity reaches each bottom bus bar 539, the second side 530 of the temperature regulating subcomponent 426 is either cooled or heated (e.g., if the first side 529 is heated, then the second side 530 is cooled, if the first side 529 is cooled, the second side 530 is heated).

Thus, providing power to the temperature regulating subcomponent 426 transfers heat from the first side 529 of the temperature regulating subcomponent 426 to the second side 530 of the temperature regulating subcomponent 426 or vice versa along heat path 506.

Accordingly, the first side 529 of each of the temperature regulating subcomponents 426 either heats or cools thereby heating or cooling the interface side 427 of the temperature regulating membrane and thereby allowing the temperature regulating subassembly to heat or cool a user of the personal temperature regulating device.

Because the electrical path 505 is metal to metal contact throughout (e.g., from bottom bus bar 539, through solder to a semiconductive member 540, through solder to top bus bar 541, through solder to another semiconductive member 540, through solder to another bottom bus bar 539, etc.) the electric path 505 experiences minimal resistivity.

Further, because the components between the semiconductive layer 533 and the first side 529 of the temperature regulating subcomponent 426 are thermally conductive, heat is efficiently transferred to or away from the first side 529. Similarly, because the components between the semiconductive layer 533 and the second side 530 of the temperature regulating subcomponent 426 are thermally conductive, heat is efficiently transferred to or away from the second side 530.

Accordingly, electricity efficiently moves along electrical path 505 and heat is efficiently transferred to or away from each side 529, 530 of the temperature regulating subcomponent.

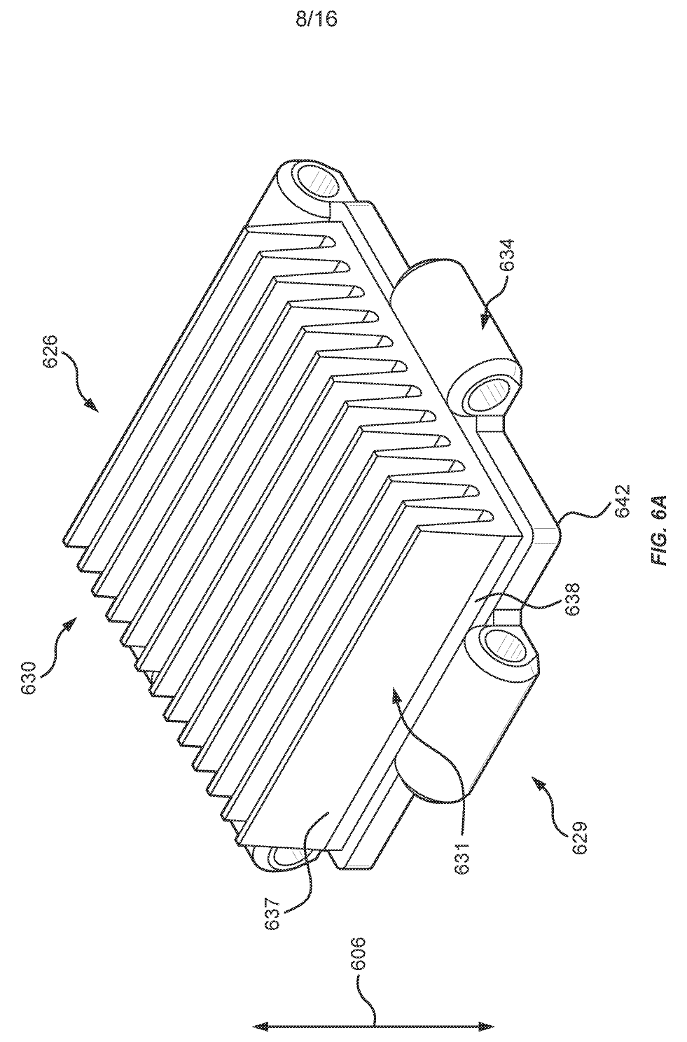

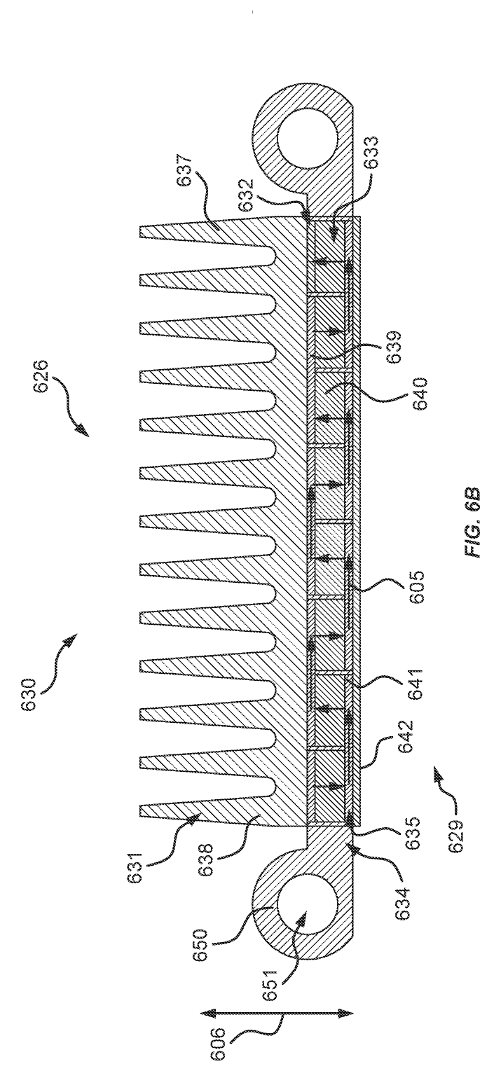

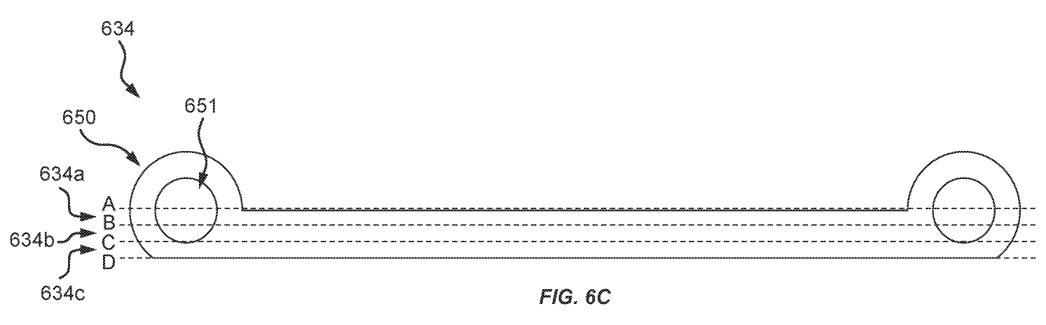

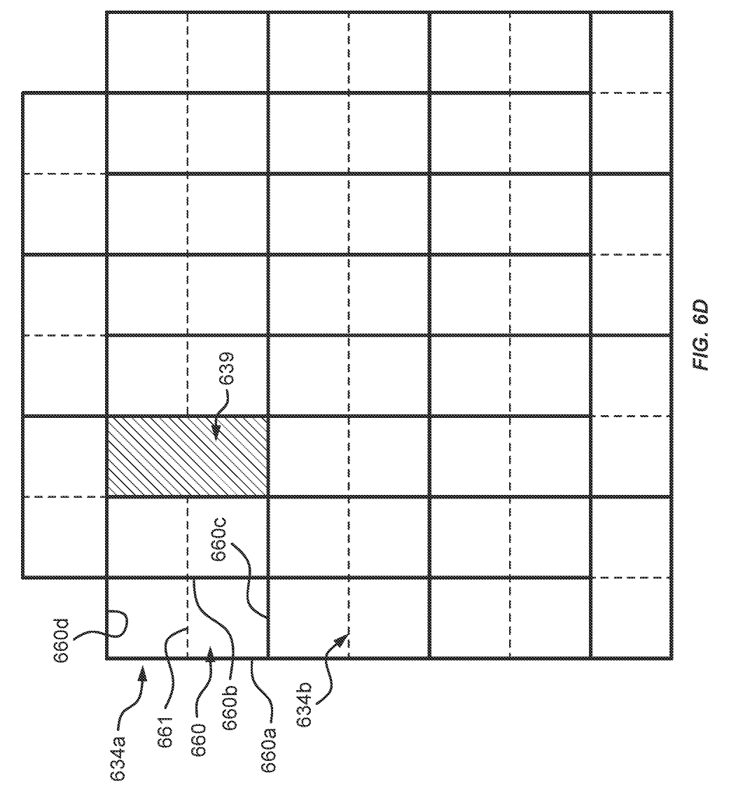

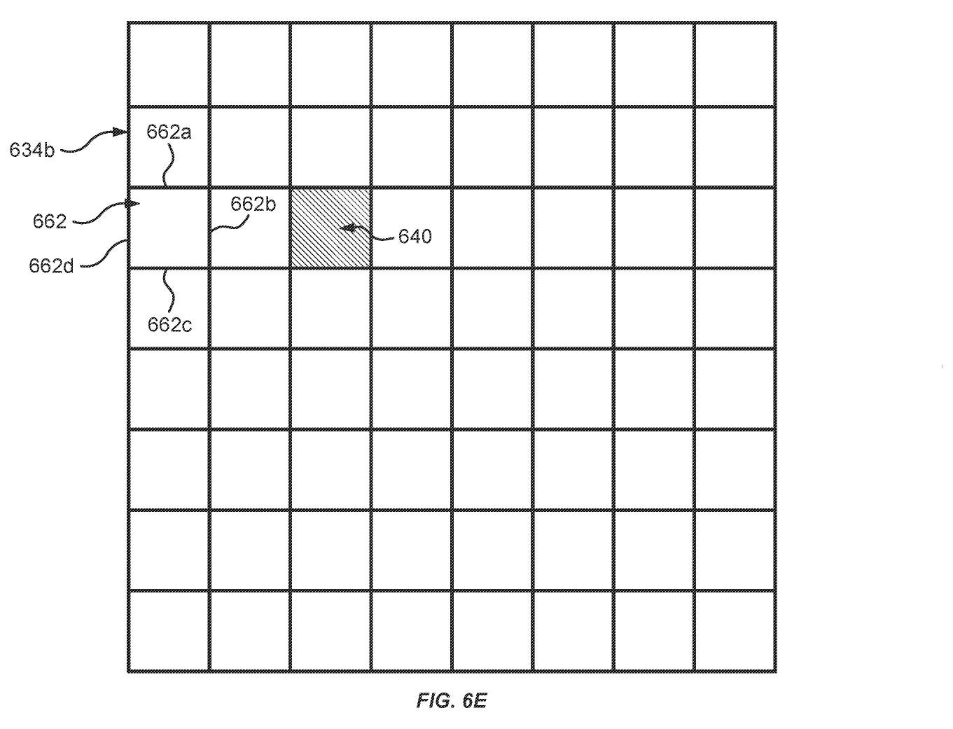

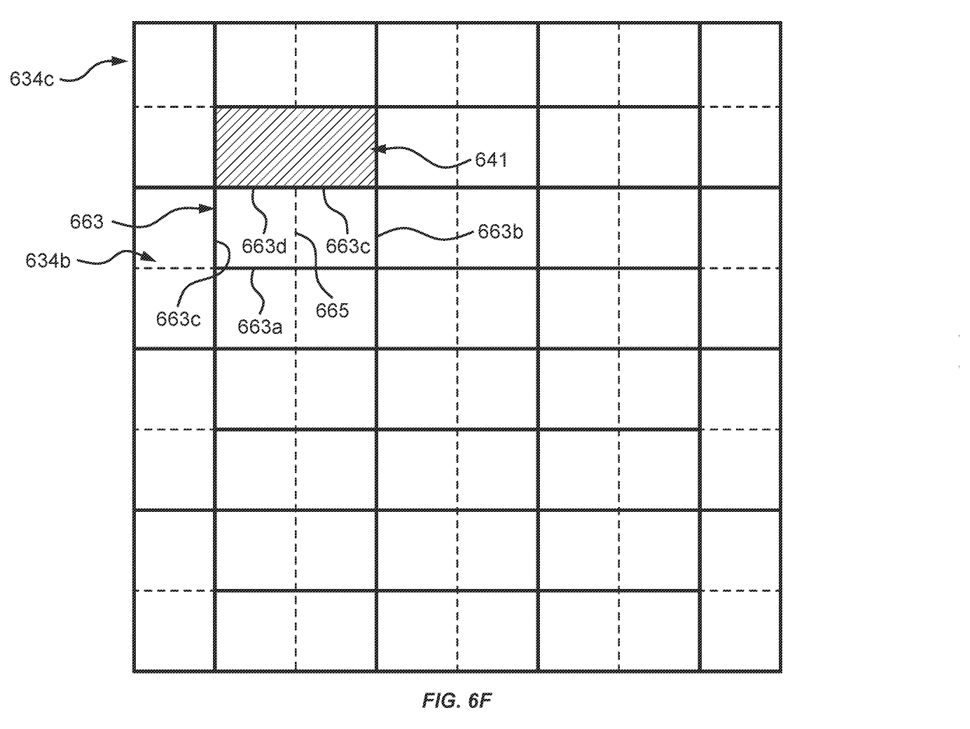

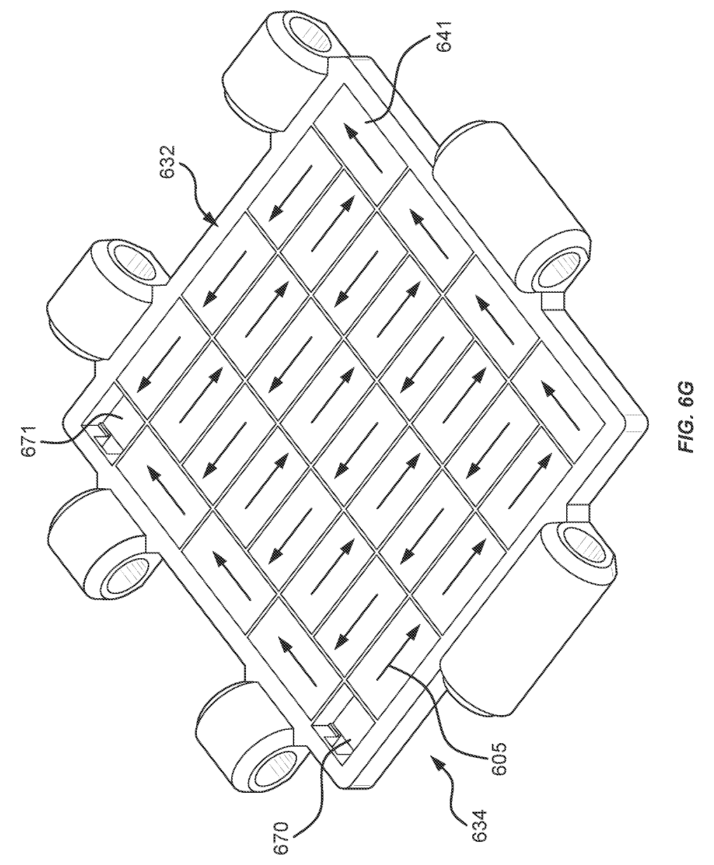

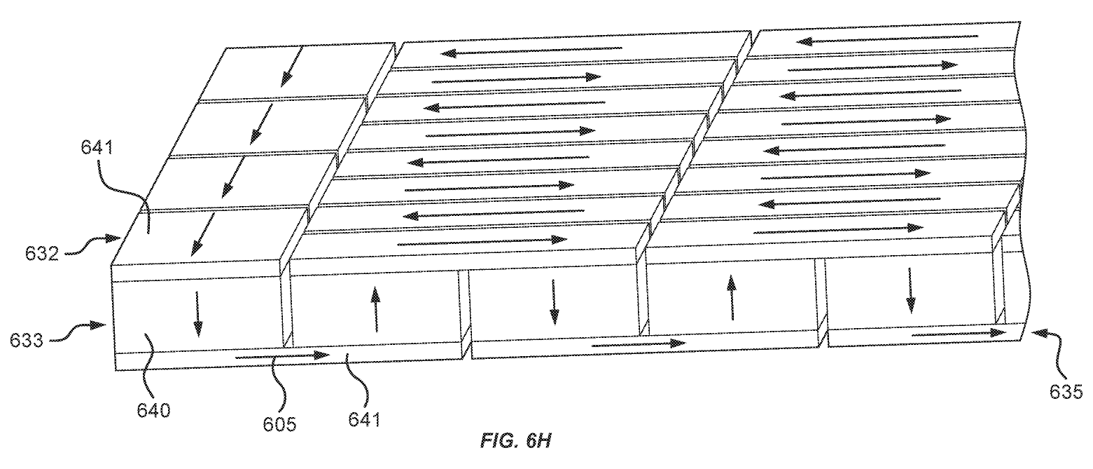

FIGS. 6A-6E illustrate another temperature regulating subcomponent 626 for use in the temperature regulating subassembly 220 of FIGS. 2-5C. Temperature regulating subcomponent 626 of FIGS. 6A-6E may include similar components to those illustrated with respect to temperature regulating subcomponent 426 of FIGS. 5A-5C. Accordingly, for the sake of brevity, like features have been given reference numerals including the same last two digits and a complete description of like features may not be repeated herein.

FIG. 6A is a perspective view of temperature regulating subcomponent 626. FIG. 6B is a cross-sectional view of the temperature regulating subcomponent 626. The temperature regulating subcomponent 626 may be a thermoelectric device. That is, the temperature regulating subcomponent 626 utilizes power supplied to the temperature regulating subcomponent 626 to transfer heat between opposite sides of the temperature regulating subcomponent 626 along heat path 606.

The temperature regulating subcomponent 626 includes a first side 629 and a second side 630. The first side 629 corresponds to an interface side (such as interface side 427 of FIGS. 4) of a temperature regulating membrane (such as temperature regulating membrane 221 of FIGS. 1-4). Thus when the first side 629 is cooled, the interface side is cooled and when the first side 629 is heated, the interface side is heated.

The temperature regulating subcomponent 626 includes a heat sink 631, a bottom bus layer 632, semiconductive layer 633, a frame 634, a top bus layer 635, and a top plate 642. The heat sink 631 is disposed on the second side 630 of the temperature regulating subcomponent 626. The heat sink 631 includes fins 637 protruding from a heat sink base 638 similar to the fins 537 of FIGS. 5A-5C. In one or more embodiments, the heat sink 631 is monolithic. That is, the heat sink base 638 and the fins 637 are monolithic. As compared to the heat sink 531 of FIGS. 5A-5C, the heat sink base 638 does not include a frame portion and rather, the base 638 includes a continuous bottom surface.

The bottom bus layer 632 is disposed below the heat sink base 638. The bottom bus layer 632 includes a plurality of bottom bus bars 639. The bottom bus bars 639 are coupled to the base 538 of the heat sink 631. In one or more embodiments, the bottom bus bars 639 are coupled by soldering or by an adhesive. In one or more embodiments, the bottom bus bars 639 are coupled to the heat sink base 638 by epoxy or glue, such as a thermal epoxy.

The stack of bottom bus layer 632, the semiconductive layer 633, and the top bus layer 635 remain unchanged from the stack of FIGS. 5A-5C and, for the sake of brevity, a description of said stack will not be repeated herein. Similarly, the top plate 642 remains unchanged from the top plate 542 of FIGS. 5A-5C and, for the sake of brevity, a description of said top plate 642 will not be repeated herein.

The frame 634 of the temperature regulating subcomponent 626 sits between the heat sink 631 and the top plate 642. The frame 634 supports and positions the bottom bus layer 632, the semiconductive layer 633, and the top bus layer 635. The frame 634 also includes one or more coupling members 650 extending from the edges of the frame 634. The coupling members 650 may include a bore 651 therethrough. The coupling members 650 are configured to interconnect the temperature regulating subcomponents 626. For example, a member, such as a flexible member (e.g., a cord, rope, piece of fabric, or elastic material 428 of FIG. 4) may be disposed through the bores 651 of the coupling members 650 of the temperature regulating subcomponents 626 to interconnect them to one another as illustrated in FIG. 4. The coupling members 650 may include varying widths and/or lengths. There may be one or more coupling members 650 per edge of the frame 634, as illustrated in FIG. 6A (e.g., two or more, three or more, four or more). Although illustrated in FIG. 6A as including six total coupling members 650 with one on two adjacent sides of the frame 634 and two on the other two adjacent sides of the frame 634, the amount and position of the coupling members 650 may be varied. For example, there may be four, five, six, seven, eight, nine, ten, or more coupling members 650 total with any amount per side.

FIG. 6C is a side view of the frame 634. The frame 634 includes a first portion 634a for supporting and positioning the bottom bus layer 632, a second portion 634b for supporting and positioning the semiconductive layer 633, and a third portion 634c for supporting and positioning the top bus layer 635. The first portion 634a is defined as the portion of the frame 634 between planes A and B. The second portion 634b is defined as the portion of the frame 634 between planes B and C. The third portion 634c is defined as the portion of the frame 634 between planes C and D. The first portion 634a, second portion 634b, and third portion 634c may be monolithic with one another (e.g., the frame 634 may be monolithic).

FIG. 6D is a top view of the first portion 634a. For illustrative purposes, the second portion 634b is shown overlayed with the first portion 634a in dashed lines. Similarly for illustrative purposes, one bottom bus bar 639 is shown disposed in the first portion 634a. The first portion 634a includes a plurality of bottom bus openings 660 forming a grid-like pattern (or an array). The bottom bus openings 660 are shaped to accommodate the outer circumference of the bottom bus bars 639 and are each defined by a first edge 660a, a second edge 660b, a third edge 660c, and a fourth edge 660d. The bottom bus openings 660 are sized to fit the bottom bus bars 639. The bottom bus openings 660 position the bottom bus bars 639. The second portion 634b of the frame 634 supports the bottom bus bars 639. That is, the second portion 634b provides cross-members 661 intersecting with the bottom bus openings 660 so that the bottom bus bars 639 are supported on one side by the cross-members 661.

FIG. 6E is a top view of the second portion 634b. The second portion 634b of the frame 634 includes a plurality of semiconductive member openings 662 forming a grid-like pattern (or an array). For illustrative purposes, one semiconductive member 640 is shown disposed in the second portion 634b. The semiconductive member openings 662 are shaped to accommodate the outer circumference of the semiconductive members 540 and each defined by a first edge 662a, a second edge 662b, a third edge 662c, and a fourth edge 662d. The semiconductive member openings 662 are sized to fit the semiconductive members 640. The semiconductive member openings 662 position the semiconductive members 640. Further, at least a portion of one or more of the first edge 662a, the second edge 662b, third edge 662c, and fourth edge 662d may be a cross member 661 for the bottom bus bars 639 as shown and described with respect to the first portion 634a in FIG. 6D.

FIG. 6F is a top view of the third portion 634c. For illustrative purposes, the third portion 634c is shown overlayed with the second portion 634b in dashed lines. Similarly for illustrative purposes, one top bus bar 641 is shown disposed in the third portion 634c. The third portion 634c includes a plurality of top bus openings 663 forming a grid-like pattern (or an array). In one or more embodiments, the pattern of the third portion 634c is similar to the first portion 634a but is clocked (e.g., rotated) 90 degrees. The top bus openings 663 are shaped to accommodate the outer circumference of the top bus bars 641 and are each defined by a first edge 663a, a second edge 663b, a third edge 663c, and a fourth edge 663d. The top bus openings 663 are sized to fit the top bus bars 641. The top bus openings 663 position the top bus bars 641. The second portion 634b of the frame 634 supports the top bus bars 641. That is, the second portion 634b provides cross-members 665 intersecting with the top bus openings 663 so that the top bus bars 641 are supported on one side by the cross-members 665. The cross-members 665 may be an edge (e.g., edges 662a-662d) of the second portion 634b.

Accordingly, when the first portion 634a, the second portion 634b, and the third portion 634c are stacked upon one another, the bottom bus bars 639 are positioned within the bottom bus openings 660 and are partially supported by the cross-members 661 and a side of the semiconductive members 640, the top bus bars 641 are positioned within the top bus openings 663 and are partially supported by the cross-members 665 and a second side of the semiconductive members 640, and the semiconductive members 640 are positioned by the semiconductive member openings 662 and are sandwiched between the top bus bars 641 and the bottom bus bars 639.

FIG. 6G illustrates a top perspective view of the frame 634 with bottom bus bars 639, the semiconductive members (not shown), and the top bus bars 641 installed. Further, FIG. 6G illustrates the electrical path 605. As shown, the electrical path 605 includes a starting point 670 and an ending point 671. The starting point 670 includes a corner of the frame 634 where there is no bottom bus bar 639 or semiconductive member (not shown). Similarly, the ending point 671 includes another corner of the frame 634 where there is no bottom bus bar 639 or semiconductive member 640.

FIG. 6H is a view of the stack of the bottom bus bars 639, semiconductive members 640, and the top bus bars 641. For illustrative purposes, the frame 634 has been removed. FIG. 6H illustrates the electrical path 605. In sum, the electrical path 605 travels across the length of a bottom bus bar 639, down (as illustrated) through a semiconductive member 640 to a top bus bar 641, across the length of a top bus bar 641, up (as illustrated) through another semiconductive member 640, and so on.

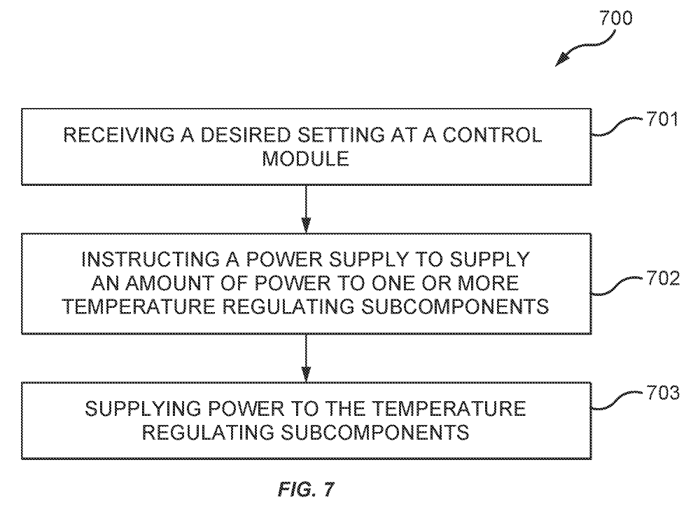

FIG. 7 illustrates a method 700 for regulating the temperature of a user (such as user 100) with a personal temperature regulating device (such as personal temperature regulating device 200 of FIG. 2).

At step 701, a desired setting is received at a control module (such as control module 223 of FIG. 2) of the personal temperature regulating device. In one or more embodiments, desired setting is set at a user interface (such as user interface 431 of FIG. 4) of the control module. The desired setting may include, but is not limited to, cooling, heating, cooling to a temperature, heating to a certain temperature, a cooling level, a heating level, a power level, or any others described herein.

At step 702 the control module transmits instructions to supply a power (such as power 404 of FIG. 4) based on the desired setting to a power supply (such as power supply 222 of FIG. 2) of the personal temperature regulating device via a transmitter (such as transmitter 432 of FIG. 4).

In one or more embodiments, a body temperature of the user is detected by one or more sensors (such as sensors 224 of FIG. 2) of the personal temperature regulating device, the detected body temperature is transmitted to the control module, and the detected body temperature is compared to the desired setting. In such embodiments, the instructions to supply the power are based on one or more of the desired setting, detected the body temperature, and/or the comparison and/or difference.

In one or more embodiments, the detected body temperature may also be compared to a body temperature safety and/or comfort threshold. In such embodiments, the instructions to supply the power are based on one or more of the desired setting, the detected body temperature, the comparison and/or difference between the desired setting and the detected body temperature, and/or the body temperature safety and/or comfort threshold.

In one or more embodiments, the surface temperature of one or more components of the personal temperature regulating device is detected by the one or more sensors, the detected surface temperature is transmitted to the control module, and the surface temperature is compared to a surface temperature safety and/or comfort threshold. In such embodiments, the instructions to supply the power are based on one or more of the desired setting, the detected body temperature, the comparison and/or difference between the desired setting and the detected body temperature, the body temperature safety and/or comfort threshold, and/or the surface temperature safety and/or comfort threshold.

At step 703, the power supply supplies an amount of power into one or more temperature regulating subcomponents (such as temperature regulating subcomponents 426 of FIG. 4) of a temperature regulating membrane (such as temperature regulating membrane 221 of FIG. 2) of the personal temperature regulating device to alter the body temperature of the user.

In one or more embodiments, the comparison between the aforementioned temperatures and/or thresholds may determine that a temperature is higher or lower than the desired setting.

The disclosure contemplates that terms such as “couples,” “coupling,” “couple,” and “coupled” may include but are not limited to, interference fitting, magnetic fastening, interlocking coupling, adhesively coupling, and pinned coupling. The disclosure contemplates that terms such as “couples,” “coupling,” “couple,” and “coupled” may include but are not limited to integrally forming. The disclosure contemplates that terms such as “couples,” “coupling,” “couple,” and “coupled” may include but are not limited to direct coupling and/or indirect coupling, such as indirect coupling through intermediate components.

Examples disclosed herein may be combined with other examples in any manner consistent with at least one of the principles disclosed herein, and references to “an example,” “some examples,” “an alternate example,” “various examples,” “one example” or the like are not necessarily mutually exclusive and are intended to indicate that a particular feature, structure, or characteristic described may be included in at least one example. The appearances of such terms herein are not necessarily all referring to the same example.

Also, the phraseology and terminology used herein is for the purpose of description and should not be regarded as limiting. Any references to examples, components, elements, acts, or functions of the embodiments shown herein referred to in the singular may also embrace embodiments including a plurality, and any references in plural to any example, component, element, act, or function herein may also embrace examples including only a singularity. Accordingly, references in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

Having described above several aspects of at least one example, it is to be appreciated various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only, and the scope of the invention should be determined from proper construction of the appended claims, and their equivalents.

It is contemplated that any one or more elements or features of any one disclosed embodiment or example may be beneficially incorporated in any one or more other non-mutually exclusive embodiments or examples. While the foregoing is directed to embodiments of the present disclosure, other and further embodiments of the disclosure may be devised without departing from the basic scope thereof, and the scope thereof is determined by the claims that follow.

The following claims are not intended to be limited to the aspects shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. §112(f) unless the element is expressly recited using the phrase “means for”. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A personal temperature regulating device, comprising:
a wearable garment; and
a temperature regulating subassembly coupled to the wearable garment, the temperature regulating subassembly comprising a temperature regulating membrane and a control module, the temperature regulating membrane comprising at least one temperature regulating subcomponent, wherein the at least one temperature regulating subcomponent is configured to alter a body temperature of a user.

2. The personal temperature regulating device of claim 1, wherein:
the at least one temperature regulating subcomponent comprises a plurality of temperature regulating subcomponents; and
the plurality of temperature regulating subcomponents are flexibly mechanically interconnected.

3. The personal temperature regulating device of claim 1, wherein:
the temperature regulating subassembly further comprises a power supply; and
the power supply is electrically coupled to the temperature regulating membrane.

4. The personal temperature regulating device of claim 1, wherein the at least one temperature regulating subcomponent comprises:
a top plate;
a heat sink disposed on a surface of the top plate;
a plurality of bottom bus bars disposed on a surface of the heat sink;
a plurality of semiconductive members disposed on a surface of the plurality of bottom bus bars, wherein each bottom bus bar of the plurality of bottom bus bars electrically couples two semiconductive members of the plurality of semiconductive members;
a plurality of top bus bars disposed on a surface the plurality of semiconductive members, wherein each top bus bar of the plurality of top bus bars electrically couples two semiconductive members of the plurality of semiconductive members; and
a frame supporting and positioning the plurality of bottom bus bars, the plurality of semiconductive members, and the plurality of top bus bars.

5. The personal temperature regulating device of claim 4, wherein:
the heat sink comprises a material, wherein the material is:
an electrically resistive material or an electrically conductive material including an electrically resistive coating; and
a thermally conductive material;
the plurality of bottom bus bars comprise an electrically conductive and thermally conductive material;
the plurality of top bus bars comprise an electrically conductive and thermally conductive material; and
the top plate comprises an electrically resistive material.

6. The personal temperature regulating device of claim 1, wherein:
temperature regulating subassembly further comprises a power supply electrically coupled to the temperature regulating membrane;
the power supply supplies an amount of power to each of the at least one temperature regulating subcomponents of the temperature regulating membrane; and
the control module controls the amount of power supplied by the power supply to each of the at least one temperature regulating subcomponents.

7. The personal temperature regulating device of claim 6, wherein:
the temperature regulating subassembly further comprises one or more temperature sensors coupled to the wearable garment;
the one or more temperature sensors are configured to detect a body temperature of the user or a surface temperature of the temperature regulating membrane; and
the control module is configured to receive the body temperature of the user or the surface temperature of the temperature regulating membrane from the one or more temperature sensors and control the amount of power supplied by the power supply to the temperature regulating membrane based on the detected body temperature or surface temperature.

8. The personal temperature regulating device of claim 1, further comprising:
a shroud, the shroud coupled to the wearable garment and at least partially covering the temperature regulating membrane.

9. The personal temperature regulating device of claim 1, the temperature regulating subassembly further comprising one or more fans coupled to the wearable garment and electrically coupled to the control module, wherein the one or more fans direct airflow over the temperature regulating membrane.

10. A temperature regulating membrane, comprising:
a plurality of temperature regulating subcomponents, wherein the plurality of temperature regulating subcomponents are flexibly mechanically interconnected, the plurality of temperature regulating subcomponents each comprising:
a top plate;
a heat sink disposed on a surface of the top plate;
a plurality of bottom bus bars disposed on a surface of the heat sink;
a plurality of semiconductive members disposed on a surface of the plurality of bottom bus bars, wherein each bottom bus bar of the plurality of bottom bus bars electrically couples two semiconductive members of the plurality of semiconductive members;
a plurality of top bus bars disposed on a surface the plurality of semiconductive members, wherein each top bus bar of the plurality of top bus bars electrically couples two semiconductive members of the plurality of semiconductive members; and
a frame supporting and positioning the plurality of bottom bus bars, the plurality of semiconductive members, and the plurality of top bus bars.

11. The temperature regulating membrane of claim 10, wherein:
the plurality of temperature regulating subcomponents are flexibly mechanically interconnected by elastic material extending between two or more temperature regulating subcomponents of the plurality of temperature regulating subcomponents.

12. The temperature regulating membrane of claim 10, wherein:
the heat sink comprises a material, wherein the material is:
an electrically resistive material or an electrically conductive material including an electrically resistive coating; and
a thermally conductive material;
the plurality of bottom bus bars comprise an electrically conductive and thermally conductive material;
the plurality of top bus bars comprise an electrically conductive and thermally conductive material; and
the top plate comprises an electrically resistive material.

13. The temperature regulating membrane of claim 10, wherein the frame of each temperature regulating subcomponent of the plurality of temperature regulating subcomponents includes one or more coupling members, and wherein the plurality of temperature regulating subcomponents are flexibly mechanically interconnected by the one or more coupling members.

14. The temperature regulating membrane of claim 10, wherein the plurality of semiconductive members are coupled to the bottom bus bars by soldering, and wherein the plurality of semiconductive members are coupled to the top bus bars by soldering.

15. The temperature regulating membrane of claim 10, wherein:
the frame includes a first frame portion, a second frame portion, and a third frame portion;
the second frame portion is disposed between the first frame portion and the second frame portion; and
the first frame portion positions the plurality of bottom bus bars, the second frame portion positions the plurality of semiconductive members, and the third frame portion positions the plurality of top bus bars.

16. The temperature regulating membrane of claim 15, wherein the frame is monolithic.

17. The temperature regulating membrane of claim 10, wherein each temperature regulating subcomponent of the plurality of temperature regulating subcomponents is generally rectangular and is about 10.0mm to about 100.0mm in length and about 10.0mm to about 100.0mm in width.

18. A method regulating the temperature of a user with a personal temperature regulating device comprising:
detecting a body temperature of a user wearing the personal temperature regulating device with a sensor of the personal temperature regulating device;
determining the body temperature is different than a setting, wherein the setting is set by a control module of the personal temperature regulating device; and
supplying an amount of power into one or more temperature regulating subcomponents of a temperature regulating membrane of the personal temperature regulating device, wherein the amount of power is based on a difference between the body temperature and the setting, and wherein the amount of power alters a surface temperature of a first side of the one or more temperature regulating subcomponents to alter the body temperature of the user.

19. The method of claim 18, wherein determining the body temperature is different than the setting comprises determining the body temperature is lower than the setting.

20. The method of claim 18, wherein determining the body temperature is different than the setting comprises determining the body temperature is higher than the setting.
